(19) United States
(12) Patent Application Publication
(10) Pub. No.: US 2018/0333075 A1
(43) Pub. Date: Nov. 22, 2018

(54) RESPIRATORY RATE MEASURING METHOD AND APPARATUS, AND WEARABLE DEVICE

(71) Applicant: SAMSUNG ELECTRONICS CO., LTD., Suwon-Si (KR)

(72) Inventors: Seung Jae Lee, Hwaseong-si (KR); Soo Yong Kim, Yongin-si (KR); Sang Shik Park, Suwon-si (KR); Yong In Park, Seoul (KR); Seoung Jae Yoo, Seongnam-si (KR)

(21) Appl. No.: 15/852,163

(22) Filed: Dec. 22, 2017

(30) Foreign Application Priority Data
May 18, 2017 (KR) 10-2017-0061468

(51) Int. Cl. A61B 5/08; A61B 5/04; A61B 5/024; A61B 5/00

(52) U.S. Cl. CPC A61B 5/0816; A61B 5/04012; A61B 5/7214; A61B 5/7235; A61B 5/02416

(57) ABSTRACT

A respiratory rate measuring method and apparatus including a wearable device that utilizes a heartbeat signal to detect the respiratory rate without motion detection of a patient's body. The method includes extracting a heartbeat signal, extracting an amplitude modulation (AM) signal and a frequency modulation (FM) signal through AM and FM with respect to the extracted heartbeat signal, respectively, performing normalization on the AM signal and the FM signal. The normalized AM signal and the normalized FM signal are combined into a single combined normalized signal, and a respiratory rate is calculated by extracting a respiratory frequency band from the combined normalized signal.

### CROSS-REFERENCE TO RELATED APPLICATION (S)

This application claims benefit of priority to Korean Patent Application No. 10-2017-0061468 filed on May 18, 2017 in the Korean Intellectual Property Office, the disclosure of which is incorporated by reference herein.

### TECHNICAL FIELD

The present inventive concept relates to a respiratory rate measuring method, a respiratory rate measuring apparatus, and a wearable device.

### DISCUSSION OF THE RELATED ART

Measuring a respiratory rate is the most basic vital sign used in determining the basic vitality of body. Various methods are used to measure a respiratory rate, such as measuring breaths per minute.

For example, spirometry is a method of measuring the air capacity of lungs by measuring a flow of air into and out of the lungs using a spirometer. Capnometry is a method of measuring a concentration or partial pressure of CO₂ in respiratory gases by breathing. Capnometry has a relatively high accuracy, but requires additional equipment and there are difficulties in requiring continuous monitoring.

On the other hand, a technique for measuring respiratory rates using a wearable-based sensor has also been proposed.

For example, impedance pneumography is a method of measuring changes in the volume of the thorax, and has high accuracy, but has a poor signal-to-noise ratio. Thus, this method has not been widely used.

In addition, although there is provided a method of estimating a respiratory rate using an acceleration sensor worn on the chest, the acceleration sensor is fixed at a point at which it may be attached and is sensitive to movement, and thus, is not suitable for continuous monitoring. Furthermore, there are negative aspects in terms of high power consumption due to the additional use of a sensor.

### SUMMARY

The present inventive concept provides a respiratory rate measuring method, a respiratory rate measuring apparatus, and a wearable device that is capable of continuously monitoring a respiratory rate with a relatively low amount of power consumption.

According to an embodiment of the present inventive concept, a respiratory rate measuring method may include the operations of extracting a heartbeat signal; extracting an amplitude modulation (AM) signal and a frequency modulation (FM) signal with respect to the heartbeat signal, respectively; normalizing the extracted AM signal and the extracted FM signal; combining the normalized AM signal and the normalized FM signal to obtain a combined normalized signal; and calculating a respiratory rate from the combined normalized signal.

According to an embodiment of the present inventive concept, a respiratory rate measuring apparatus includes a signal processor configured to extract from a heartbeat signal, an amplitude modulation (AM) signal and a frequency modulation (FM) signal through AM and FM with respect to the heartbeat signal, respectively, normalizing the AM signal and the FM signal and then combining the normalized AM and FM signals, and calculating a respiratory rate from the combined signal; and an output unit configured to output the respiratory rate calculated by the signal processor.

According to an embodiment of the present inventive concept, a wearable device includes a wearable sensor configured for attachment to a user's body to measure a heartbeat signal; and at least one processor configured to extract from the heartbeat signal an amplitude modulation (AM) signal and a frequency modulation (FM) signal, through AM and FM with respect to the heartbeat signal measured by the wearable sensor, respectively, the at least one processor further configured to normalize the AM signal and the FM signal and then combining the normalized AM and FM signals, and to calculate a respiratory rate from the combined normalized AM and normalized FM signal.

According to an embodiment of the inventive concept, the respiratory frequency band extracted from a separated principal component signal ranges from about 0.1 Hz to 0.7 Hz.

According to an embodiment of the inventive concept, the measuring of the respiratory rate is based on only the heartbeat signal of a patient without motion detection of a patient.

### BRIEF DESCRIPTION OF THE DRAWINGS

The present inventive concept will be better understood and appreciated by a person of ordinary skill in the art from the following detailed description when taken in conjunction with the accompanying drawings, in which:

FIG. 1 is a flowchart of a respiratory rate measuring method according to an example embodiment of the present inventive concept;

FIGS. 2A, 2B, 2C and 2D are diagrams illustrating respective examples of heartbeat signals extracted according to an example embodiment of the present inventive concept, and AM and FM signals extracted therefrom, in which:

FIG. 2A illustrates a heartbeat signal extracted according to an example embodiment of, for example, an electrocardiogram signal;

FIG. 2B illustrates an electrocardiogram signal in which baseline wandering occurs;

FIG. 2C illustrates an AM signal extracted through amplitude modulation on the electrocardiogram signal; and

FIG. 2D illustrates an FM signal extracted through frequency modulation on the electrocardiogram signal;

FIGS. 3A and 3B are diagrams illustrating respective examples of two signals before normalization according to an example embodiment of the present inventive concept;

FIGS. 4A and 4B are diagrams illustrating respective examples of two signals such as shown in FIG. 3A and FIG. 3B after normalization according to an example embodiment of the present inventive concept is performed;

FIG. 5 is a block diagram of a respiratory rate measuring apparatus according to an example embodiment of the present inventive concept;

FIG. 6 is a block diagram of a respiratory rate measuring apparatus according to an example embodiment of the present inventive concept; and

FIG. 7 is a drawing illustrating a wearable device according to an example embodiment of the present inventive concept.

### DETAILED DESCRIPTION

Hereinafter, example embodiments of the present inventive concept will be described with reference to the accompanying drawings. A person of ordinary skill in the art should understand and appreciate that the inventive concept as recited in the appended claims is not limited to the example embodiments shown herein.

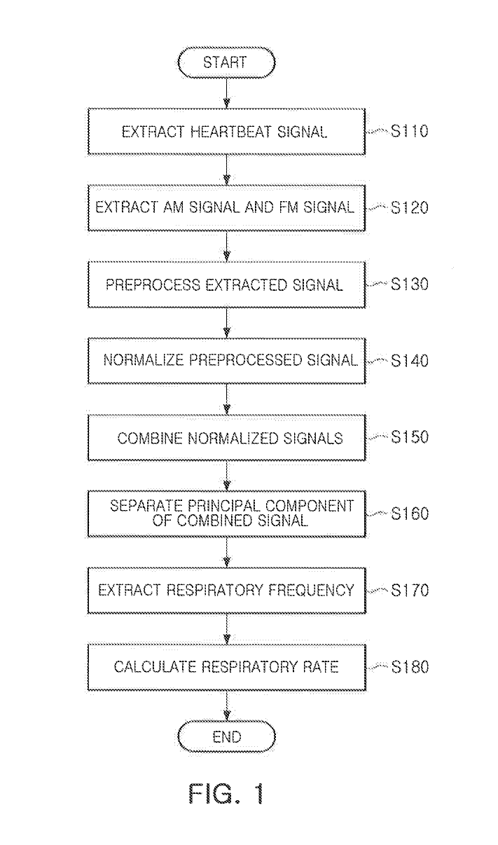

FIG. 1 is a flowchart of a respiratory rate measuring method according to an embodiment of the present inventive concept.

Referring to the example shown FIG. 1, at operation S110, a heartbeat signal may be extracted to measure a respiratory rate.

For example, the extracted heartbeat signal may have been extracted by various types of equipment, including but not limited to one of an electrocardiogram signal measured using an electrocardiography (ECG) device and a pulse wave signal measured through a photoplethysmography (PPG) sensor.

However, the type of equipment used to extract a heartbeat signal in an example embodiment of the present inventive concept is not limited thereto, and a variety of heartbeat signals known in the art may be used. For example, as described below with regard to the example embodiments, the signal used to extract a heartbeat signal is a signal capable of extracting an amplitude modulation (AM) signal and a frequency modulation (FM) signal through AM and FM with respect to the heartbeat signal.

In the case, for example, of an ECG, the electrical activity of the heart (e.g. heartbeat signal), is a substantially periodic wave that can include an AM component, an FM component and an additive component. As the respiratory activity impacts the ECG, an extraction of an AM signal and an FM signal, for example, can be used to determine a respiratory rate.

In addition, according to an embodiment of the inventive concept, the heartbeat signal may be the sole basis for calculating a respiratory rate in a wearable device (e.g. no use of accelerator sensors to detect movement as in a respiratory rate estimation method based on movement of the thorax) to determine respiration activity.

Subsequently, at operation 120, the AM signal and the FM signal may be extracted through the amplitude modulation and the frequency modulation on the heartbeat signal, respectively.

In this example, the amplitude modulation is a modulation scheme of changing an amplitude of a given signal. The frequency modulation is a modulation scheme of changing a frequency in proportion to a signal magnitude while allowing an amplitude of the signal to be constant. Since the amplitude modulation and frequency modulation schemes are techniques known in the art, a detailed description thereof will be omitted.

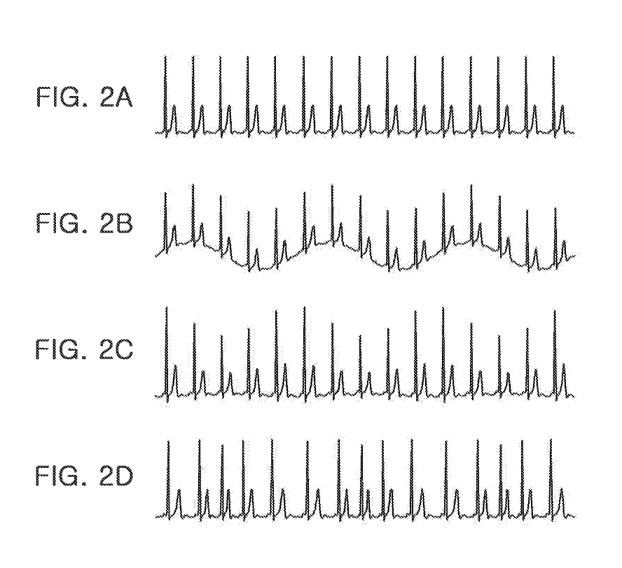

FIGS. 2A to 2D are diagrams illustrating examples of heartbeat signals extracted according to an example embodiment, and AM and FM signals extracted therefrom.

In more detail, FIG. 2A illustrates an extracted heartbeat signal according to an example embodiment of the present disclosure, for example, an electrocardiogram signal, and FIG. 2B illustrates an electrocardiogram signal in which baseline wandering (e.g., a drift of the baseline) occurs. FIG. 2C illustrates an AM signal extracted through amplitude modulation of the electrocardiogram signal, and FIG. 2D illustrates an FM signal extracted through frequency modulation of the electrocardiogram signal.

In the case of baseline wandering, the baseline variation illustrated in FIG. 2B may occur if there is a problem with an attachment point of an electrode for extraction of a heartbeat signal, and the baseline wandering may be removed through a baseline variation removal algorithm or the like in preprocessing to be described herein after.

Subsequently, preprocessing on the extracted modulation signals, for example, the AM signal and the FM signal, may be performed in S130, as required.

For example, in the preprocessing process, preprocessing techniques may be performed, including noise cancellation, interpolation, DC offset cancellation and the like on respective modulation signals. A noise cancellation technique and an interpolation technique used in the preprocessing process on modulation signals may be employed in various technologies known in the art, and a detailed description thereof will be omitted.

Then, normalization may be performed on respective preprocessed modulation signals in S140.

In this case, the normalization is an operation to match a range of data or make distribution similar.

According to an example embodiment, one way to combine the AM signal and the FM signal extracted from the heartbeat signal may include that a normalization process be performed, in which energy levels of the two signals become similar to each other, as described subsequently herein.

For example, the normalization of respective modulation signals may be performed according to Equation 1:

$$Normalized = \frac{Original}{RMS power} .$$

In Equation 1, ‘Normalized’ represents a normalized signal, ‘Original’ represents a preprocessed modulation signal, and ‘RMS power’ may be calculated according to Equation 2:

$$RMS = \sqrt{\frac{{x}_{1}^{2} + {x}_{2}^{2} + … + {x}_{n}^{2}}{n}} .$$

The normalization method described above is provided by way of example. Thus, various normalization methods known in the art may be may be used.

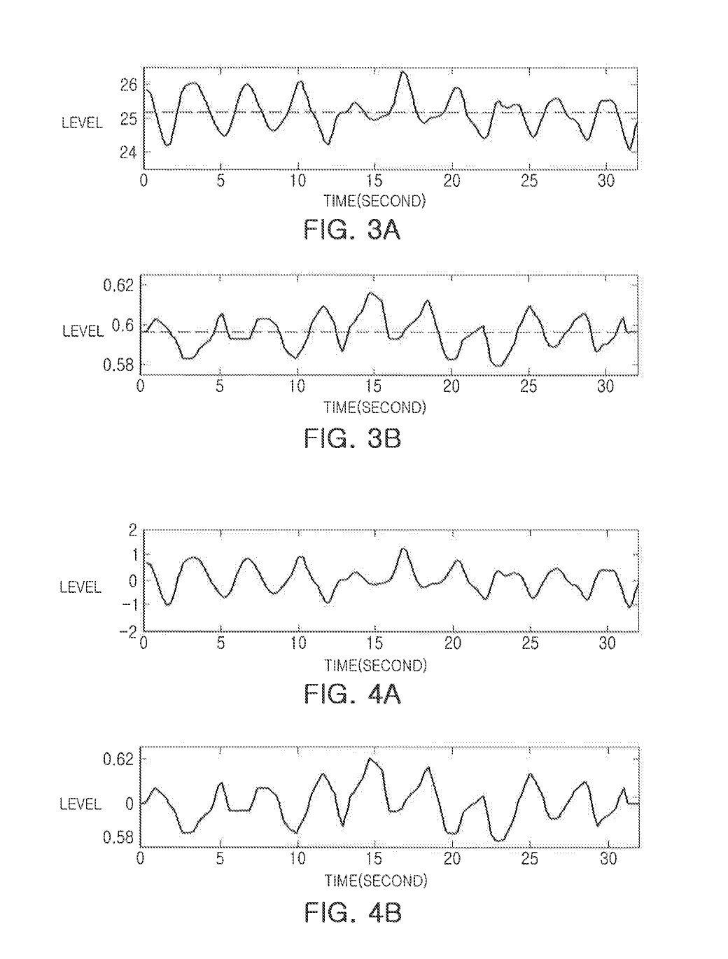

FIGS. 3A and 3B are diagrams illustrating examples of two signals prior to normalization occurring according to an example embodiment of the inventive concept. FIGS. 4A and 4B are diagrams illustrating examples of two signals after normalization occurring according to an example embodiment of the inventive concept.

As illustrated in FIGS. 3A and 3B, for example, when two signals having different levels are subjected to a normalization process, two signals having a similar distribution level may be obtained as illustrated in FIGS. 4A and 4B.

Then, two normalized modulation signals, for example, the AM signal and the FM signal, may be combined in S150.

For example, convolution may be used to combine the normalized AM and FM signals to form a third signal, which may be referred to as an impulse response.

The AM and FM signals, which are normalized through convolution as described herein above, may be combined to extract a common frequency from the respective modulation signals, and a respiration information may be extracted through the common frequency.

In addition, weights of the AM signal and the FM signal may be adjusted, based on input information, to combine two signals. When the weights are adjusted, the input information may be a unique factor affecting the heartbeat signal. For example, the input information may include age, gender, and the like of a user, and two signals may be combined by using predetermined weights of predetermined AM and FM signals according to input information.

According to the example embodiment described above, as the common frequency is extracted from a signal obtained by combining the normalized AM signal and the normalized FM signal, the inventive concept provides for extraction of more accurate respiration activity without the use of additional sensors to detection motion activity (e.g., motion sensors to detection motion of the thorax).

In addition, a respiratory rate may be calculated from the combined signal. For example, a principal component of the combined signal, which may be a frequency component related to respiration, may be separated from the combined signal (FIG. 1, operation S160), and a respiration frequency may be extracted from a separate signal (FIG. 1, operation S170), thereby calculating the respiratory rate in S180.

In an example, a frequency band of, for example, 0.1 Hz to 0.7 Hz, which is associated with respiration, may be extracted from the separate signal, and a spectral peak may be detected therefrom, and based thereon, a respiratory rate, breaths/min, may be calculated.

A respiratory rate measuring method described above with reference to FIG. 1 may be performed using hardware, such as at least one processor, a MicroController Unit (MCU), and the like.

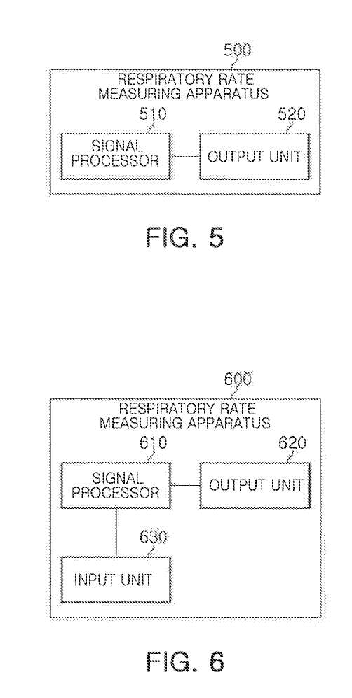

FIG. 5 is a block diagram of a respiratory rate measuring apparatus according to an example embodiment of the inventive concept.

With reference to FIG. 5, a respiratory rate measuring apparatus 500 according to this embodiment may include a signal processor 510 (e.g., signal processing unit) and an output unit 520.

The signal processor 510 may calculate a respiratory rate by analyzing a received heartbeat signal.

In detail, the signal processor 510 may extract an AM signal and an FM signal through amplitude modulation and frequency modulation on a heartbeat signal, respectively, normalize the extracted AM and FM signals, combine the normalized AM and FM signals, and calculate a respiratory rate from the combined signal.

In addition, the signal processor 510 may further perform preprocessing on respective modulation signals.

The signal processor 510 may extract a principal component of the combined signal and extract a respiration frequency from the separate signal to calculate the respiratory rate.

With continued reference to FIG. 5, a detailed method of analyzing the heartbeat signal to calculate the respiratory rate by the signal processor 510 is substantially similar to that described above with reference to FIG. 1, and thus, overlapping descriptions thereof will may be omitted.

For example, the output unit 520 may be configured to output a respiratory rate calculated by the signal processor 510. The output unit 520 may be embodied by a display device displaying information, a communications module configured to transmit information, and the like, to output or transmit information regarding a calculated respiratory rate.

FIG. 6 is a block diagram of a respiratory rate measuring apparatus according to an example embodiment of the present inventive concept.

With reference to FIG. 6, a respiratory rate measuring apparatus 600 according to an example embodiment may further include an input unit 630, in addition to the configuration of the respiratory rate measuring apparatus 500 illustrated in FIG. 5.

The input unit 630 may be configured to receive information from a user, and for example, may receive information including a unique factor affecting a heartbeat signal.

A signal processor 610 may be configured to adjust weights of respective modulation signals when normalized as AM and FM signals, based on the information input through the input unit 630, are combined.

Thus, the signal processor 610 may separate a principal component from a combined signal by applying the adjusted weights thereto, and may extract a respiration frequency from the separate signal, to calculate a respiratory rate.

The respiratory rate measuring apparatuses 500 and 600 described above with reference to FIGS. 5 and 6 may be implemented by hardware, such as a processor, an MCU, or the like, or may be implemented in an application form to be installed in a user terminal, such as a smartphone, a tablet PC, or the like.

In addition, the respiratory rate measuring apparatuses 500 and 600 may be connected to a sensor outputting a heartbeat signal, such as an ECG sensor, a PPG sensor, or the like, in a wired or wireless manner, to a processor to analyze the heartbeat signal received from the sensor and calculate a respiratory rate.

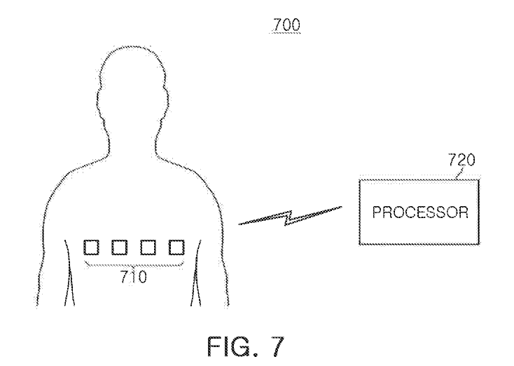

FIG. 7 is a drawing illustrating a wearable device according to an example embodiment of the inventive concept.

With reference to FIG. 7, a wearable device 700 according to an example embodiment may include a wearable sensor 710 and at least one processor 720.

The wearable sensor 710 may be configured to attach to the body of a user to measure a heartbeat signal, and for example, may include a sensor outputting a heartbeat signal, such as an ECG sensor, a PPG sensor, or the like.

According to an example embodiment of the inventive concept, the wearable sensor 710 may be implemented as a patch type sensor configured to be attached to one or more points of the body, and the wearable sensor 710, when worn by the user, is configured to measure and output the heartbeat signal of the user.

The at least one processor 720 may be configured to analyze the heartbeat signal output from the wearable sensor 710 and to calculate a respiratory rate.

More particularly, the at least one processor 720 may be configured to extract an AM signal and an FM signal through amplitude modulation and frequency modulation on a heartbeat signal, respectively, normalize the extracted AM and FM signals, combine the normalized AM and FM signals, and calculate a respiratory rate from a combined signal.

In addition, the at least one processor 720 may further perform preprocessing on the respective modulation signals.

With further reference to FIG. 7, the at least one processor 720 may calculate the respiratory rate by separating a principal component of the combined signal and extracting a respiration frequency from the separate signal.

A detailed method of analyzing the heartbeat signal to calculate the respiratory rate by the at least one processor 720 is identical to that described above with reference to FIG. 1, and thus, overlapping descriptions thereof will be omitted.

The wearable sensor 710 and the at least one processor 720 may be separated from each other and may be connected to each other by wired or wireless communications, or may also be integrally coupled and implemented as a single patch-type chip.

As set forth above, in a respiratory rate measuring method according to an example embodiment according to the inventive concept, a respiratory rate may be measured using only a heartbeat signal and without using an additional sensor. Thus, a respiratory rate may be monitored in a relatively less complicated manner, and may be continuously monitored with relatively low power consumption.

In addition, in implementing a wearable device, the wearable device may be variously designed without limitations to positions in which the wearable device may be attached to the body. By permitting the wearable device to have various positions at which there may be attachment to a user will enhance the convenience of the wearable device.

While example embodiments of the inventive concept have been shown and described above, a person of ordinary skill in the art should understand and appreciate that modifications and variations could be made without departing from the scope of the present inventive concept as defined by the appended claims.

1. A respiratory rate measuring method comprising:
extracting a heartbeat signal;
extracting an amplitude modulation (AM) signal and a frequency modulation (FM) signal with respect to the heartbeat signal, respectively;
normalizing the extracted AM signal and the extracted FM signal;
combining the normalized AM signal and the normalized FM signal to obtain a combined normalized signal; and
calculating a respiratory rate from the combined normalized signal.

2. The respiratory rate measuring method of claim 1, wherein the heartbeat signal is one of an electrocardiogram signal measured by an electrocardiography device or a pulse wave signal measured by a photoplethysmography sensor.

3. The respiratory rate measuring method of claim 1, wherein the combining of the normalized AM signal and the normalized FM signal is performed by convolution.

4. The respiratory rate measuring method of claim 1, wherein the combining of the normalized AM signal and the normalized FM signal includes adjusting weights of the normalized AM signal and the normalized FM signal, based on input information on a factor affecting the heartbeat signal.

5. The respiratory rate measuring method of claim 4, wherein the adjusting weights of the normalized AM signal and the normalized FM signal is performed with a predetermined weight with respect to the normalized AM signal and the normalized FM signal according to the input information.

6. The respiratory rate measuring method of claim 4, wherein the input information comprises at least one of an age and gender of a patient.

7. The respiratory rate measuring method of claim 1, wherein the calculating of the respiratory rate from the combined normalized signal is performed by separating a principal component from the combined normalized signal into a separated signal, and extracting a respiratory frequency band from the separated signal.

8. The respiratory rate measuring method of claim 7, wherein the respiratory frequency band extracted from the separated signal ranges from about 0.1 Hz to 0.7 Hz.

9. The respiratory rate measuring method of claim 1, further comprising performing preprocessing on the extracted AM signal and the extracted FM signal prior to performing the normalization of the extracted AM signal and the extracted FM signal.

10. A respiratory rate measuring apparatus comprising:
a signal processor configured to extract an amplitude modulation (AM) signal and a frequency modulation (FM) signal with respect to a heartbeat signal received thereby, respectively, and to normalize the extracted AM signal and the extracted FM signal and combine the normalized AM signal and the normalized FM signal into a combined normalized signal, and calculate a respiratory rate from the combined normalized signal; and
an output unit configured to output the respiratory rate calculated by the signal processor.

11. The respiratory rate measuring apparatus of claim 10, wherein the measuring of the respiratory rate is based on only the heartbeat signal of a patient without motion detection of the patient.

12. The respiratory rate measuring apparatus of claim 10, wherein the signal processor combines the normalized AM signal and the normalized FM signal by performing a convolution operation.

13. The respiratory rate measuring apparatus of claim 10, further comprising an input unit receiving input information regarding at least one factor that affects the received heartbeat signal.

14. The respiratory rate measuring apparatus of claim 13, wherein the signal processor adjusts weights of the AM signal and the FM signal to combine the normalized AM signal and the normalized FM signal, based on the input information.

15. The respiratory rate measuring apparatus of claim 10, wherein the signal processor is configured to separate a principal component of the combined normalized signal into a separated signal, and extract a respiratory frequency from the separated signal to calculate the respiratory rate.

16. The respiratory rate measuring apparatus of claim 10, wherein the signal processor is configured to perform preprocessing on the extracted AM signal and the extracted FM signal prior to the extracted AM signal and the extracted FM signal being normalized.

17. A wearable device comprising:
a wearable sensor configured for attachment to a patient's body to measure a heartbeat signal; and
at least one processor configured to extract an amplitude modulation (AM) signal and a frequency modulation (FM) signal with respect to a heartbeat signal output from the wearable sensor, respectively, and to normalize the AM signal and the FM signal and combine the normalized AM signal and the normalized FM signal into a combined normalized signal, and to calculate a respiratory rate from the combined normalized signal.

18. The wearable device of claim 17, wherein the wearable sensor comprises an electrocardiography device that senses an electrocardiogram signal or a photoplethysmography sensor that senses a pulse wave signal.

19. The wearable device of claim 17, wherein the wearable sensor is configured to be attached as a patch-type sensor to one or more regions of the patient's body.

20. The wearable device of claim 17, wherein the at least one processor is configured to adjusts weights of the AM signal and the FM signal, based on input information about a factor affecting the heartbeat signal, and is configured to combine the adjusted AM and FM signals.

21. (canceled)

22. (canceled)
